(19) United States
(12) Patent Application Publication
(10) Pub. No.: US 2005/0049305 A1
(43) Pub. Date: Mar. 3, 2005

(54) Use of L-carnitine in the prevention and treatment of hearing loss

(76) Inventors: Gilda M. Kalinec, Playa del Rey, CA (US); Federico Kalinec, Playa del Rey, CA (US); David J. Lim, Pasadena, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/899,439

(22) Filed: Jul. 26, 2004

Related U.S. Application Data
(63) Continuation of application No. PCT/US03/02832, filed on Jan. 30, 2003.
(60) Provisional application No. 60/353,200, filed on Jan. 30, 2002.

(51) Int. Cl. A61K 31/205; A61K 31/22

(52) U.S. Cl. 514/546; 514/554

(57) ABSTRACT

Preferred embodiments of the current invention relate to methods of preventing and/or treating hearing loss by administration of L-carnitine or its derivatives. More particularly, administration of L-carnitine or its derivatives is disclosed for preventing and/or treating hearing loss caused to mothers and/or their offspring during the perinatal period by the ototoxic effects of chemotherapeutic agents, aminoglycoside antibiotics, as well as excess noise.

### RELATED APPLICATIONS

This Application is a continuation of International Patent Application PCT/US03/002832 filed Jan. 30, 2003 designating the US and published in English as WO 2003/063789 on Aug. 7, 2003, which claims the benefit of priority of U.S. Provisional Patent Application No. 60/353,200 filed Jan. 30, 2002, both of which are expressly incorporated herein by reference in their entireties.

### BACKGROUND OF THE INVENTION

1. Field of the Invention

Preferred embodiments of the current invention relate to methods of preventing and/or treating hearing loss by administration of L-carnitine or its derivatives.

2. Description of the Related Art

Hearing loss to an extent sufficient to interfere with social and job-related communications is among the most common chronic neural impairments in the United States. It is estimated that more than 28 million Americans have hearing impairment and that as many as 2 million are profoundly deaf. The annual cost of lost productivity, special education, and medical treatment is estimated at over $30 billion for hearing, speech and language disorders.

In many cases, the cause of vestibular disorders is likely to lie in the inner ear. Balance disorders increase in frequency in the older age groups and, by age 75, this disorder becomes one of the most common reasons for seeking help from physicians. According to the National Ambulatory Medical Care Survey for 1991, dizziness-vertigo is among the 25 most common reasons Americans visit the doctor. U.S. physicians report a total of more than 5 million dizziness-vertigo visits a year. The annual cost of medical care for patients with balance disorders is estimated to exceed $1 billion in the United States. These figures will likely increase with the aging of the baby boomer generation.

Two references disclose the otoprotective effects of acetyl-L-carnitine (ALCAR), the predominant acylated ester of L-carnitine (LCAR). Coleman et al. 2001 hypothesize that ALCAR attenuates noise-induced hearing loss by acting as an antioxidant on reactive oxygen species produced due to metabolic exhaustion of the inner ear from loud continuous noise exposure. Seidman et al. 2000 attribute the otoprotective effects of ALCAR to its protection of cochlear mitochondrial DNA from deletions caused by reactive oxygen species, a phenomenon associated with aging.

LCAR is a natural micronutrient required for normal mitochondrial function. It is essential to the importation of activated long-chain fatty acids across the mitochondrial membrane, modulation of the intramitochondrial acetyl-coenzyme A (CoA)/free-CoA ratio, modulation of the Krebs cycle and ATP formation, and scavenging of potentially toxic compounds before they have a chance to accumulate inside the mitochondrial matrix.

It has been proposed that LCAR and its esters protect many cell types from oxidative damage, both by inhibiting free-radical propagation and by contributing to the repair of oxidized membrane phospholipids. Mitochondria constitute the greatest source of oxidants in a mammalian cell. A mitochondrion is an organelle which produces ATP, and its electron transport system consumes approximately 85% of the oxygen utilized by the cell. The multiplicative effects of LCAR in reversing the decline in various physiological parameters associated with mitochondrial function may be attributable to its ability to deliver acetyl-CoA equivalents to the tricarboxylic acid cycle and to facilitate the mitochondrial β-oxidation of fatty acids, thereby increasing the production of ATP. The β-oxidation of fatty acids serves as an essential source of energy for many tissues. It is plausible that LCAR can increase the metabolic efficiency of compromised populations of mitochondria and cause a redistribution of the metabolic workload, resulting in increased cellular efficiency and a decrease in the rate at which mitochondria-derived oxidants are produced.

LCAR homeostasis is maintained through a combination of absorption from dietary sources, biosynthesis, and renal reabsorption. Dietary LCAR provides more than half of the available LCAR in humans, and is absorbed by an active transport process across the human proximal small intestinal mucosa.

LCAR is not considered an essential nutrient for adult humans only because it can be synthesized, from the essential amino acids of lysine and methionine. Lysine provides the carbon chain and nitrogen atom of LCAR, and methionine provides the methyl group. The rate of LCAR biosynthesis in humans is approximately 2 μmol/kg/day and does not appear to fluctuate significantly. The essentially constitutive nature of LCAR biosynthesis is thought to be due to a relatively constant low rate of lysine availability from the turnover of various proteins that contain this amino acid. It is believed that substrate availability rather than the activity of the pathway enzymes is the determinant in LCAR biosynthesis. Therefore, the only way LCAR biosynthesis could be increased would be through increased methylation of protein-bound lysine and/or increased protein turnover. Thus, the ability of humans to adapt to changes in the need for LCAR is severely restricted, consequently increasing dependence on exogenous LCAR in conditions of increased demand.

Under physiological conditions, plasma LCAR concentration is maintained within a narrow range. The concentration of free carnitine (LCAR and its esters) in plasma of human adults is about 35-40 μM. The level is lower in strict vegetarians or lacto-ovo-vegetarians. The dominant factors in maintaining plasma LCAR concentration are the amount of dietary LCAR and the amount of LCAR eliminated by renal clearance. In humans, approximately 54-87% of dietary LCAR is absorbed, 90-98% of the LCAR filtered by the kidney is reabsorbed, and the remainder is excreted as metabolites in urine and feces. However, if the normal plasma LCAR concentration is increased—e.g., by ingestion of a large amount of LCAR, or by infusion of LCAR into the blood stream—the rate of LCAR excretion increases disproportionately, and the efficiency of LCAR reabsorption decreases. These observations suggest that the efficiency of reabsorption contributes significantly to LCAR homeostasis by modulating the concentration of carnitine in plasma.

Certain health conditions associated with an increased prevalence of hearing loss have been linked with LCAR deficiency—e.g., renal failure, AIDS, premature birth, low birth weight, cancer and cisplatin chemotherapy.

Exogenous LCAR may be critical during pregnancy. LCAR levels in pregnant women diminish significantly in the late stages of pregnancy, with plasma concentration equivalent to LCAR deficient conditions (<20 μM). Although the cause or causes responsible for this LCAR depletion are not known, it is suspected that part is derived to the fetus and part to the uterus, that develops from an organ of approximately 50 g to a muscular mass over 2 kg. It has also been suggested that LCAR may play a role in removing potentially toxic acyl groups from the cells of the fetus and the mother, which must be excreted as acylcarnitine into urine. This would explain the increase in renal clearance observed in pregnant women and the increased need of LCAR during the pregnancy. In pregnant women, LCAR is secreted by and transferred across the placenta, which provides significant stores to the growing fetus essentially during the third trimester of gestation. Fetuses at the last stages of gestation and newborn infants depend heavily on lipids as a concentrated source of fuel to achieve rapid growth. As mentioned, LCAR is essential to the importation of activated long-chain fatty acids across the mitochondrial membrane.

Skeletal muscle LCAR concentration is positively correlated with gestational age at birth—at 25 weeks of gestation the skeletal muscle LCAR concentration is less than half that at 42 weeks gestation. Fetuses and preterm neonates have impaired reabsorption of LCAR and ALCAR at the level of the proximal tubes, which matures with advancing gestational age. Moreover, LCAR synthesis at birth (in humans) is about 12% of that in adults because of the very low activity of the hepatic gamma-butyrobetaine-hydroxylase, an enzyme crucial for the synthesis of LCAR. The combination of increased demand, decreased synthesis capacity, insufficient stores and increased losses by the immature renal tubule renders the fetuses and preterm neonates strictly dependent on the exogenous supplies to maintain a normal plasma LCAR concentration.

A variety of commonly used drugs have ototoxic properties. The best known are aminoglycoside antibiotics such as gentamicin and streptomycin, loop diuretics, salicylates and anti-tumor chemotherapeutic agents such as cisplatin. Ototoxicity has also been described during oral or parenteral administration of erythromycin. Most ototoxic agents cause hearing loss and/or balance disorders by damaging the sensory hair cells or the stria vascularis, a specialized epithelial organ responsible for the homeostasis of electrolytes within the inner ear. While there are hypotheses, little is currently known about the biochemistry (on either a cellular or a molecular level) of drug ototoxicity.

### SUMMARY OF THE INVENTION

The object of the various embodiments of the present invention is to provide methods of treating or preventing hearing loss with L-carnitine (LCAR) and its esters, such as acetyl-L-carnitine (ALCAR). A method of preventing or treating a hearing loss induced in a pregnant mammal and/or its offspring by exposure to an ototoxic agent during the perinatal period is disclosed. The method comprises administering to the pregnant mammal and/or its offspring during the perinatal period an amount of a pharmaceutical composition comprising L-carnitine or a derivative thereof. The amount of the pharmaceutical composition is sufficient to prevent or treat the hearing loss induced by exposure to the ototoxic agent. Specific embodiments counteract the ototoxicity of chemotherapeutic agents, such as cisplatin. Other specific embodiments counteract the ototoxicity of aminoglycoside antibiotics, such as gentamicin and streptomycin. Yet other specific embodiments prevent noise-induced hearing loss as well as neonatal mortality. In one preferred embodiment the mammal is pregnant. In another preferred embodiment the mammal is a fetus. In another preferred embodiment the mammal is a newborn. One preferred embodiment prevents apoptosis of sensory cells in the organ of Corti. Another preferred embodiment prevents apoptosis of sensory cells in the vestibular organ. Certain embodiments comprise the administration of pharmaceutical compositions comprising L-carnitine or derivatives thereof. In other embodiments, the derivative of L-carnitine is an ester. Some of these embodiments comprise the administration of acetyl-L-carnitine. In one preferred embodiment the administration is prior to an exposure to an ototoxic agent. In another preferred embodiment the administration is concurrent with an exposure to an ototoxic agent. In yet another preferred embodiment the administration is after an exposure to an ototoxic agent.

### BRIEF DESCRIPTION OF THE DRAWINGS

FIG. 1 is a graph of auditory brainstem response (ABR) measurements which demonstrate an increase in hearing threshold induced by cisplatin, and an attenuation of this increase by L-carnitine (LCAR) supplementation. A, ABR waveforms in newborn guinea pigs in three groups—control, cisplatin, and cisplatin+LCAR. B, a comparison of average ABR thresholds (**P≦0.01 over control group). C, an analysis of variance of the treatment (with Treatment and Age) indicating that cisplatin's noxious effect was more significant in newborns (***P≦0.003) that in adults (P≦0.07)

FIG. 2 is a series of confocal microscopy images showing an increase in outer hair cell (OHC) damage in organ of Corti of newborn guinea pigs induced by cisplatin, and a preventive effect by LCAR supplementation against this damage. A, Control; B, Cisplatin (cell damage was observed in all three rows of OHCs); C, OHC damage was significantly prevented by LCAR supplementation.

FIG. 3 is a graphical representation of Table 1, showing the percentage increase in outer hair cell damage induced by cisplatin, and a preventive effect by LCAR supplementation against this damage.

FIG. 4 is a graph of auditory brainstem response (ABR) measurements which demonstrate an increase in hearing threshold induced by gentamicin, and an attenuation of this increase by LCAR supplementation.

FIG. 5 is a bar graph showing that gentamicin significantly increases hearing thresholds in mothers and newborn guinea pigs, and that this increase is attenuated by LCAR supplementation.

FIG. 6 is a series of images from scanning electron microscopy showing gentamicin-induced cochlear damage. A, full cochlea; B, Control; C and D, gentamicin.

FIG. 7 is a series of images from confocal microscopy showing gentamicin-induced disruption of stereocilia bundle in OHCs, and the preventive effect of LCAR. A, Control; B, gentamicin; C, pre-treatment with LCAR; D, co-treatment with LCAR.

FIG. 8 is a series of images from confocal (A, B, C) and scanning electron microscopy (SEM) (A′, B′, C′) showing noise-induced cochlear damage in organ of Corti. A and A′—Control; B and B′—noise-exposed; C and C′—high magnification images of OHCs hair bundles from noise-exposed animals.

FIG. 9 is a graph of vestibular organ cell (HEI-Ve1) apoptosis induced by: penicillin, gentamicin, streptomycin, and cisplatin.

FIG. 10 is a graph of organ of Corti cell (HEI-OC1) apoptosis induced by: penicillin, gentamicin, streptomycin, and cisplatin.

FIG. 11 is a graph showing an increase in organ of Corti cell (HEI-OC1) apoptosis induced by cisplatin and gentamicin, and the attenuation of this increase by LCAR supplementation. ***P≦0.001, **P≦0.01 (with respect to Control).

FIG. 12 is a graph of mouse fibroblast cell (NIH3T3) apoptosis induced by: penicillin, gentamicin, streptomycin, and cisplatin. NIH3T3 mouse fibroblasts are used as a control against which the results in FIGS. 9 and 10 are viewed.

### DETAILED DESCRIPTION OF THE PREFERRED EMBODIMENT

Preferred modes of the present invention are based upon the surprising discovery that LCAR and its derivatives are able to both prevent and treat perinatal damage to sensory hair cells. As shown herein, administration of LCAR and its derivatives prevents damage to sensory hair cells and/or reduces damage to sensory hair cells or cochlear neurons during the perinatal period. Moreover, LCAR and its derivatives also significantly reduce noise- and ototoxin-induced neonatal mortality.

In one embodiment, the present invention provides a method for preventing damage to sensory hair cells or cochlear neurons in a subject by administering an effective amount of LCAR or its derivatives. In another aspect, the present invention provides a method for treating existing damage to sensory hair cells in a subject by administering a therapeutically effective amount of LCAR or its derivatives. Also disclosed herein are methods of preventing neonatal mortality induced by ototoxic agents by administering a therapeutically effective amount of LCAR or its derivatives.

In general, the following words or phrases have the indicated definition when used in the description, examples, and claims. The terms “preventing” or “treating” are used herein in the context of hearing loss, loss of sense of balance, death of sensory hair cells or cochlear neurons, sensorineural hearing loss, or damage to sensory hair cells or cochlear neurons and the like, to mean reducing, minimizing, or completely eliminating such loss or damage. An object of both prophylactic and therapeutic measures is to prevent or diminish, respectively, neuron-damage-related hearing impairment, such as ototoxin-induced damage. Those in need of LCAR treatment in accordance with the present invention include those already experiencing a hearing impairment, those prone to having the impairment, and/or those in which exposure to an ototoxic insult or drug is predicted or deliberate (e.g., cancer patient about to begin chemotherapy with an ototoxic drug) and the resultant impairments are to be prevented.

The hearing impairments may be due to hair cell or neuronal damage, wherein the damage is caused by loud sounds or chemical-induced ototoxicity. Ototoxins may include therapeutic drugs such as antineoplastic agents, salicylates, quinines, and aminoglycoside antibiotics, as well as contaminants in foods or medicinals, and environmental or industrial pollutants.

In a preferred embodiment of the present invention, LCAR treatment is undertaken to prevent or reduce ototoxicity, which is expected to result from administration of ototoxic therapeutic drugs. Preferably, a therapeutically effective formulation comprising LCAR is given shortly after exposure to an ototoxin to prevent or reduce the ototoxic effect. More preferably, the LCAR treatment is provided prophylactically, either by administration of the LCAR formulation prior to or concomitantly with the ototoxic drug or the exposure to the ototoxic insult. As used herein, “preventing” and “treating” may include, for example, at least about a 15% reduction of loss or damage, more preferably at least about 25%, more preferably at least about 50%, even more preferably at least about 75%, even more preferably at least about 80%, even more preferably at least about 85%, even more preferably at least about 90%, even more preferably at least about 95%, and most preferably about 100%.

As used herein, the term “sensory hair cells” refers to the hair cells present in vertebrates, including the auditory sensory hair cells present in the Organ of Corti, and the vestibular sensory hair cells present in the semicircular canals and maculae of the inner ear.

As used herein, the term “hearing loss” refers to a reduced ability to perceive auditory stimuli that are perceivable by a normally functioning subject.

As used herein, the term “loss of sense of balance” refers to a deficit in the vestibular system of an animal compared to the vestibular system of a normally functioning subject.

As used herein, the term “death of sensory hair cells” refers to a cessation of the ability of one or more sensory hair cells in perceiving and/or transducing sensory stimuli.

By “ototoxic agent” in the context of the present invention is meant a substance that through its chemical or mechanical action, injures, impairs, or inhibits the activity of a component of the nervous system related to hearing, thereby resulting in impair hearing. The list of ototoxic agents that cause hearing impairments includes, but is not limited to, neoplastic agents such as vincristine, vinblastine, cisplatin, taxol, or dideoxy-compounds, e.g., dideoxyinosine; alcohol; metals; industrial toxins involved in occupational or environmental exposure (like toluene); contaminants of food or medicinals; or over-doses of vitamins or therapeutic drugs, e.g., antibiotics such as penicillin or chloramphenicol, or megadoses of vitamins A, D, or B6, salicylates, quinines and loop diuretics. Loud noise is also meant to be included in the list of ototoxic agents. By “exposure to an ototoxic agent” is meant that the ototoxic agent is made available to, or comes into contact with a mammal. Exposure to an ototoxic agent can occur by direct administration, e.g., by ingestion or administration of a food, medicinal, or therapeutic agent, e.g., a chemotherapeutic agent, by accidental contamination, or by environmental exposure, e.g., aerial or aqueous exposure. Fetal exposure may also occur systemically when a drug given the mother crosses the placental barrier.

“Chronic” administration refers to administration of the agent(s) in a continuous mode as opposed to an acute mode, so as to maintain the initial anti-ototoxic effect for an extended period of time.

“Mammal” refers to any animal classified as a mammal, including humans, domestic and farm animals, and zoo, sports, or pet animals, such as dogs, horses, cats, cows, etc. Preferably, the mammal herein is human.

“Perinatal” relates to the period immediately before and after birth (and includes the mother and its offspring). As used herein, the perinatal period in humans starts from about 6 month before to 6 month after birth.

A “patient” for the purposes of the present invention includes both humans and other mammals. Thus the methods are applicable to both human therapy and veterinary applications. In preferred embodiments, the mammal may be pregnant, a fetus or a newborn.

The term “administration” includes but is not limited to, oral, subbuccal, transdermal, parenteral, intravenous, subcutaneous and topical. A common requirement for these routes of administration is efficient and easy delivery of LCAR and its derivatives to the target.

One mode of administration of LCAR and its derivatives is oral. LCAR and its derivatives may be administered orally to a subject in a number of ways, including, but not limited to tablets, capsules and caplets.

Another mode of administration of LCAR and its derivatives to the subject is subbuccal through the use of tablets.

Yet another mode of administration of LCAR and its derivatives is subcutaneous administration.

Another mode of administration contemplated by the present invention is topical. LCAR and its derivatives may be administered topically in a number of ways, including, as a cream, a lotion, a patch, an ointment, as aerosol sprays, or as drops, including but not limited to eardrops and nosedrops.

The nature of the pharmaceutical composition for the administration is dependent on the mode of administration and can readily be determined by one of ordinary skill in the art. For example, for oral administration, pharmaceutical compositions may contain, in addition to LCAR and its derivatives, pharmaceutically acceptable carriers, vehicles, buffers and excipients.

As used herein, the term “effective amount”, refers to the amount of LCAR and its derivatives required to achieve an intended purpose for both prophylaxis or treatment without undesirable side effects, such as toxicity, irritation or allergic response. Although individual needs may vary, the determination of optimal ranges for effective amounts of formulations is within the skill of the art. Human doses can readily be extrapolated from animal studies (Katocs et al., 1990 Chapter 27 In: Remington's Pharmaceutical Sciences, 18th Ed., Gennaro, ed., Mack Publishing Co., Easton, Pa.). Generally, the dosage required to provide an effective amount of a formulation, which can be adjusted by one skilled in the art, will vary depending on several factors, including the age, health, physical condition, weight, type and extent of the disease or disorder of the recipient, frequency of treatment, the nature of concurrent therapy, if required, and the nature and scope of the desired effect(s) (Nies et al. 1996 Chapter 3 In: Goodman & Gilman's The Pharmacological Basis of Therapeutics, 9th Ed., Hardman et al., eds., McGraw-Hill, New York, N.Y.).

By “hearing impairment” is meant a neurologic disorder, oto-neurological in nature, typically sensorineural, but including composite loss (both sensorineural and conductive loss), preferably either a sensory or a neural hearing loss, and most preferably a sensory loss (cochlear related), in which the patient will display, complain of, or is diagnosed to have a hearing loss. Conductive hearing loss is typically related to the external or middle ear. These impairments of interest to the present invention are those associated with damage, loss, or degeneration of a sensory cell of the auditory system. Preferably such impairments can occur along with neuronal damage or conductive hearing loss damage. The loss can be unilateral. Hair cells are epithelial cells possessing fine projections and located in the organ of Corti (auditory system) or the vestibular system.

Hearing impairments relevant to the invention may be sensory hearing loss due to end-organ lesions. Hearing impairments include tinnitus, which is a perception of sound in the absence of an acoustic stimulus, and may be intermittent or continuous, wherein there is diagnosed a sensorineural loss. The hearing loss can be congenital, such as that caused by ototoxic drugs administered to the mother. The hearing loss can be noise-induced, generally due to a noise greater than 85 decibels (dB) that damages the inner ear. Alternatively, the hearing loss may be caused by an ototoxic drug that effects the auditory portion of the inner ear, particularly the organ of Corti. Incorporated herein by reference are Chapters 196, 197, 198 and 199 of The Merck Index, 14th Edition, (1982), Merck Sharp & Dome Research Laboratories, N.J. and related chapters in the most recent edition) relating to description and diagnosis of hearing impairments.

Tests are known and available for diagnosing hearing impairments. Neuro-otological, neuro-ophthalmological, neurological examinations, and electro-oculography can be used. (Wennmo et al. 1982 Acta Otolaryngol 94:507-15). Sensitive and specific measures are available to identify patients with auditory impairments. For example, tuning fork tests can be used to differentiate a conductive from a sensorineural hearing loss and determine whether the loss is unilateral. An audiometer is used to quantitate hearing loss, measured in decibels. With this device the hearing for each ear is measured, typically from 125 to 8000 Hz, and plotted as an audiogram. Speech audiometry can also be performed. The speech recognition threshold, the intensity at which speech is recognized as a meaningful symbol, can be determined at various speech frequencies. Speech or phoneme discrimination can also be determined and used an indicator of sensorineural hearing loss since analysis of speech sounds relies upon the inner ear and 8th nerve. Tympanometry can be used to diagnose conductive hearing loss and aid in the diagnosis of those patients with sensorineural hearing loss. Electrocochleography, measuring the cochlear microphonic response and action potential of the 8th nerve, and evoked response audiometry, measuring evoked response from the brainstem and auditory cortex, to acoustic stimuli can be used in patients, particularly infants and children or patients with sensorineural hearing loss of obscure etiology. These tests serve a diagnostic function as well as a clinical function in assessing response to therapy.

Sensory and neural hearing losses can be distinguished based on tests for recruitment (an abnormal increase in the perception of loudness or the ability to hear loud sounds normally despite a hearing loss), sensitivity to small increments in intensity, and pathologic adaptation, including stapedial reflex decay.

In one embodiment, the invention constitutes a method for treating a mammal having or prone to a hearing impairment or treating a mammal prophylactically to prevent or reduce the occurrence or severity of a hearing impairment that would result from exposure to a hair cell injury, loss, or degeneration, such as that caused by an ototoxic agent, wherein a therapeutically effective amount of LCAR and/or its derivatives is administered to the mammal. Preferably the derivative is an ester form of LCAR, more preferably an acetyl-L-carnitine. Optionally, LCAR, its derivatives, or functional analogues are administered alone or in combination. By “functional analogues” we mean compounds that have different chemical formulae than LCAR and its derivatives, but cause similar effects as LCAR and its derivatives, and work through common mechanisms of action. Additional optional components of an LCAR formulation in accordance with the present invention include a hair cell growth factor or agonist, which are compounds known to promote hair cell survival, regeneration, growth, proliferation, or prevent or reduce cytotoxicity of hair cells. Additional constituents may also include, but are not limited to, promoters of mitochondrial function, facilitators of oxidative phosphorylation and substances that combat oxidative stress. The compositions and methods of the invention in preventing or treating hearing impairment are particularly effective when such impairment is induced by an ototoxic agent.

It is another object of the invention to provide a method for treating a mammal to prevent, reduce, or treat a hearing impairment, preferably an ototoxic agent-induced hearing impairment, by administering to a mammal in need of such treatment a composition containing a prophylactically or therapeutically effective amount of LCAR and/or its derivatives in combination with a prophylactically or therapeutically effective amount of an agent that acts synergistically or additively to enhance or complement the prophylactic or therapeutic effect of LCAR and/or its derivatives.

In one embodiment, a method is disclosed for treating a hearing impairment wherein the ototoxicity results from administration of a therapeutically effective amount of an ototoxic pharmaceutical drug. Typical ototoxic drugs are chemotherapeutic agents, e.g., antineoplastic agents, and antibiotics. Other possible candidates include loop-diuretics, quinines or a quinine-like compounds, and salicylate or salicylate-like compounds.

The methods of the invention are particularly effective when the ototoxic compound is an antibiotic, such as for example, an aminoglycoside antibiotic. Ototoxic aminoglycoside antibiotics include but are not limited to neomycin, paromomycin, ribostamycin, lividomycin, kanamycin, amikacin, tobramycin, viomycin, gentamicin, sisomicin, isepamicin, netilmicin, streptomycin, dibekacin, fortimicin, and dihydrostreptomycin, or combinations thereof. The antibiotics include the several structural variants of the above compounds (e.g., kanamycin A, B and C; gentamicin A, C1, C 1a, C2 and D; neomycin B and C and the like). The free bases, as well as pharmaceutically acceptable acid addition salts of these aminoglycoside antibiotics are also candidate ototoxic agents within the broad meaning of that term.

Hearing impairments induced by aminoglycosides can be prevented or reduced by the methods of the invention. Although the aminoglycosides are powerful antibiotics which are employed against bacterial infections caused by susceptible organisms, their usefulness tends to be restricted to more severe, complicated infections, because of their ototoxic and nephrotoxic side-effects. For this reason the aminoglycosides are considered to have a low therapeutic/risk ratio compared to other antibiotics used systemically. However, use of LCAR and/or its derivatives in combination with such ototoxic antibiotics increases their therapeutic/risk ratio by reducing the risk of ototoxic damage at therapeutically effective doses of the antibiotic.

For the purpose of this disclosure, the terms “pharmaceutically acceptable acid addition salt” shall mean a mono or poly salt formed by the interaction of one molecule of the aminoglycoside antibiotic with one or more moles of a pharmaceutically acceptable acid. Included among those acids are acetic, hydrochloric, sulfuric, maleic, phosphoric, nitric, hydrobromic, ascorbic, malic and citric acid, and those other acids commonly used to make salts of amine-containing pharmaceuticals.

Accordingly, the methods and compositions of the invention find use for the prevention and treatment of opportunistic infections in animals and humans which are immunosuppressed as a result of either congenital or acquired immunodeficiency or as a side effect of chemotherapeutic treatment. According to an alternate embodiment of the present invention, LCAR and/or its derivatives are used advantageously in combination with a known antimicrobial agent to provide improved methods and compositions to prevent and/or treat diseases induced by gram positive bacteria including, but not limited to: Staphylococcus aureus, Streptococcus pneumonia, Hemophilus influenza; gram negative bacteria including, but not limited to: Escherichia coli; Bacterium enteritis, Francisella tularensis; acid-fast bacteria including, but not limited to Mycobacterium tuberculosis, and Mycobacterium leprae.

In some embodiments LCAR and/or its derivatives are co-administered with an ototoxic agent. For example, an improved method is provided for treatment of infection of a mammal by administration of an aminoglycoside antibiotic, the improvement comprising administering a therapeutically effective amount of LCAR and/or its derivatives to the patient in need of such treatment to reduce or prevent ototoxin-induced hearing impairment associated with the antibiotic. In yet another embodiment, an improved method for treatment of cancer in a mammal is provided, wherein administration of a chemotherapeutic compound is combined with administration of a therapeutically effective amount of LCAR and/or its derivatives to the patient in need of such treatment, thereby preventing or reducing the ototoxin-induced hearing impairment associated with the chemotherapeutic drug.

Also provided herein are methods for preventing neonatal mortality due to exposure to an ototoxic agent by administering of the LCAR or its derivatives or functional analogues prior, during, or after such exposure.

Also provided herein are methods for preventing cell apoptosis upon, prior to, or after exposure to an agent or effect that is capable of inducing a sensorineural hearing impairment. Such agents and effects are those described herein. The method includes the step of administering to the cell an effective amount of LCAR, its derivatives, and functional analogues, or other compositions containing same as discussed herein. Preferably, the method is used upon, prior to, or after exposure to a hearing-impairing ototoxic agent.

In one embodiment the methods of treatment are applied to hearing impairments resulting from the administration of a chemotherapeutic agent to treat its ototoxic side effect. Ototoxic chemotherapeutic agents amenable to the methods of the invention include, but are not limited to an antineoplastic agent, including cisplatin or cisplatin-like compounds, taxol or taxol-like compounds, and other chemotherapeutic agents believed to cause ototoxin-induced hearing impairments, e.g., vincristine, an antineoplastic drug used to treat hematological malignancies and sarcomas.

In one embodiment LCAR and/or its derivatives is administered prior to administration or exposure to a hearing-impairing event such as exposure to an ototoxic agent.

In another embodiment LCAR and/or its derivatives is administered with an agent that promotes hair cell growth, proliferation, regeneration, or survival.

An effective amount of LCAR and/or its derivatives to be employed therapeutically will depend, for example, upon the therapeutic objectives, the route of administration, the species of the patient, and the condition of the patient. Accordingly, it will be necessary for the therapist to titer the dosage and modify the route of administration as required to obtain the optimal therapeutic effect. As is known in the art, adjustments for age as well as the body weight, general health, diet, time of administration, drug interaction and the severity of the disease may be necessary, and will be ascertainable with routine experimentation by those skilled in the art. A typical daily dosage of LCAR and/or its derivatives used alone might range from about 1 μg/kg to up to 500 mg/kg of patient body weight or more per day, depending on the factors mentioned above, preferably about 10 μg/kg/day to 100 mg/kg/day. Typically, the clinician will administer LCAR and/or its derivatives until a dosage is reached that repairs, maintains, and, optimally, reestablishes cell function to relieve the hearing impairment. The progress of this therapy is easily monitored by conventional assays and neurological diagnostic methods.

LCAR and/or its derivatives optionally is combined with or administered in concert with ototoxic pharmaceutical drugs. Initially the drugs are administered in conventional therapies known for the ototoxic pharmaceutical. Adjustments to the therapies are at the discretion of the skilled therapist to titrate dosages and conditions that decrease ototoxicity-related hearing while maintaining, and preferably improving, treatment outcomes with the ototoxic pharmaceutical drug.

Accordingly, methods for preventing or reducing ototoxicity of an aminoglycoside antibiotic or other ototoxic pharmaceutical are disclosed herein, which comprise the administration of an effective dose of LCAR and/or its derivatives. In addition, provided herein are compositions having reduced ototoxicity as a result of incorporation of the ototoxicity-inhibiting LCAR and/or its derivatives of the present invention. These pharmaceutical compositions comprise an effective ototoxicity-inhibiting amounts of LCAR and/or its derivatives as described herein, therapeutically effective amounts of the ototoxic pharmaceutical drug, e.g., aminoglycosides antibiotic, anti-neoplastic agent such as cisplatin, and optionally a pharmaceutically acceptable carrier and/or vehicle which would be familiar to one skilled in the pharmaceutical arts. The actual amounts of ototoxic pharmaceutical drug employed will range from those given in standard references for prescription drugs, e.g., Physicians Desk Reference, Medical Economics Data Production Co., Montvale, N.J. (1995), “Drug Evaluations” AMA, 6th Edition (1986); to amounts somewhat larger since the ototoxicity potential is reduced in these compositions.

The effective amounts of such agents, if employed, will be at the physician's or veterinarian's discretion. Dosage administration and adjustment is done to achieve the best management of hearing (and when used in conjunction with an ototoxic pharmaceutical drug, the indication for the ototoxic drug). The dose will additionally depend on such factors as the type of drug used and the specific patient being treated. Typically the amount employed will be the same dose as that used if the drug were to be administered without agonist; however, lower doses may be employed depending on such factors as the presence of side-effects, the condition being treated, the type of patient, and the type of agonist and drug, provided the total amount of agents provides an effective dose for the condition being treated.

The effectiveness of treating hearing impairments with the methods of the invention can be evaluated by the following signs of recovery, including recovery of normal hearing function, which can be assessed by known diagnostic techniques including those discussed herein, and normalization of nerve conduction velocity, which is assessed electrophysiologically.

The following examples illustrate the present invention. The examples do not limit the present invention in any way. A person skilled in the art can perform alternative ways which are still in the scope of the present invention.

### EXAMPLES

Guinea pigs were used as an animal model to evaluate whether LCAR supplementation during pregnancy exerts a protective effect against a series of ototoxic challenges, including gentamicin and streptomycin (both aminoglycoside antibiotics), cisplatin (an anti-tumor chemotherapeutic agent) and noise. In guinea pigs, like in humans, the period of maximum cochlear development occurs during the last stages of pregnancy.

### LCAR Supplementation Prevents Cisplatin-Induced Sensorineural Hearing Loss.

Pregnant guinea pigs were divided in three groups of 4 each. One week after delivery, those in groups #1 (3 mothers & 9 newborns—one mother died during the experiment) and #2 (4 mothers & 15 newborns) received an intraperitoneal injection of cisplatin (4 mg/kg body mass) once a day for 2 consecutive days. Those from group #2 also received a supplementary dose of LCAR in their drinking water (1 mg/ml ad libitum˜100 mg/kg/day) during the second half of the pregnancy and the immediate postnatal period. Guinea pigs from group #3 (4 mothers & 10 newborns), the control group, were injected with saline solution. Two weeks after delivery, all guinea pigs (mothers and newborns) were anesthetized by intraperitoneal injections of ketamine hydrochloride (60 mg/kg) and xylazine hydrochloride (5 mg/kg), and their auditory brainstem responses (ABR) were measured. They were then euthanized with CO₂, and potential cochlear damage was evaluated by confocal microscopy.

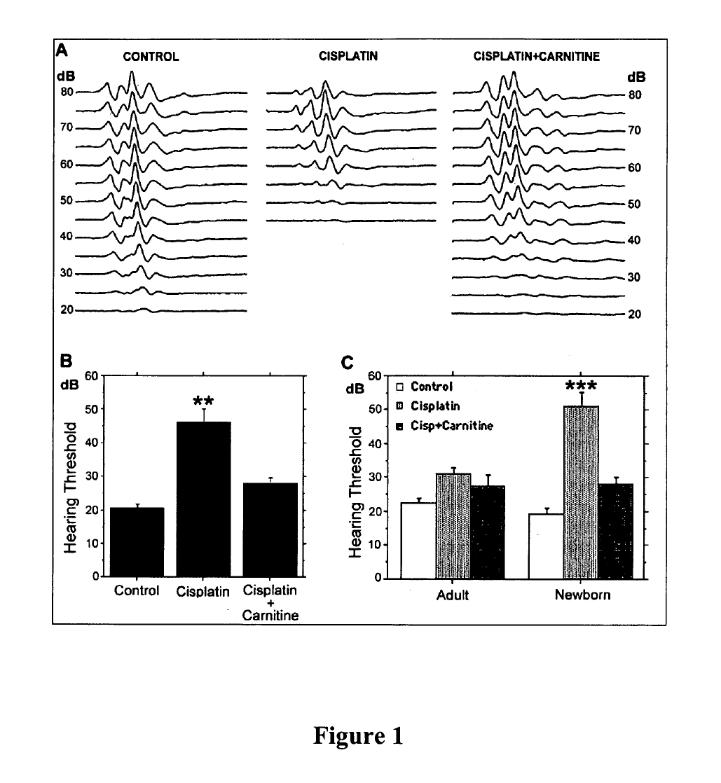

The ABR recordings in FIG. 1 show a significant cisplatin-induced increase in hearing threshold in both mothers and newborns. Data was statistically analyzed by 3-way analysis of variance, with treatment (control, cisplatin or cisplatin+carnitine), age (mothers or newborns) and ear (left or right) as independent factors. Cisplatin induced a significant threshold shift (46±4 dB vs. 21±2 dB in the control group; P≦0.01, FIG. 1B). In contrast, group 2 (cisplatin+LCAR) presented threshold values similar to those measured in control animals (28±2 dB vs. 21±2 dB, P≦0.13, FIG. 1B). The effect of cisplatin was more pronounced in newborns than in their mothers (51±4 dB vs. 31±2 dB; P≦0.003, FIG. 1C), while the left and right ears were affected alike (31±3 dB vs. 37±3 dB; P≦0.09). All the expressed values correspond to mean ±S.E.M. These results clearly indicate a protective role of LCAR against the noxious effects of cisplatin on hearing function.

### LCAR Supplementation Prevents Cisplatin-Induced Cochlear Damage

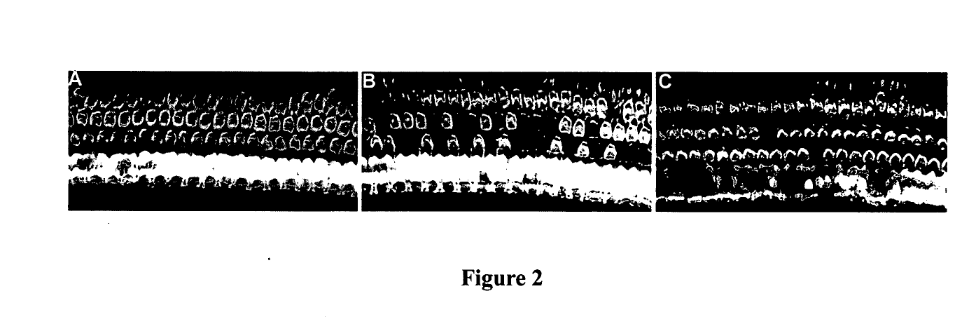

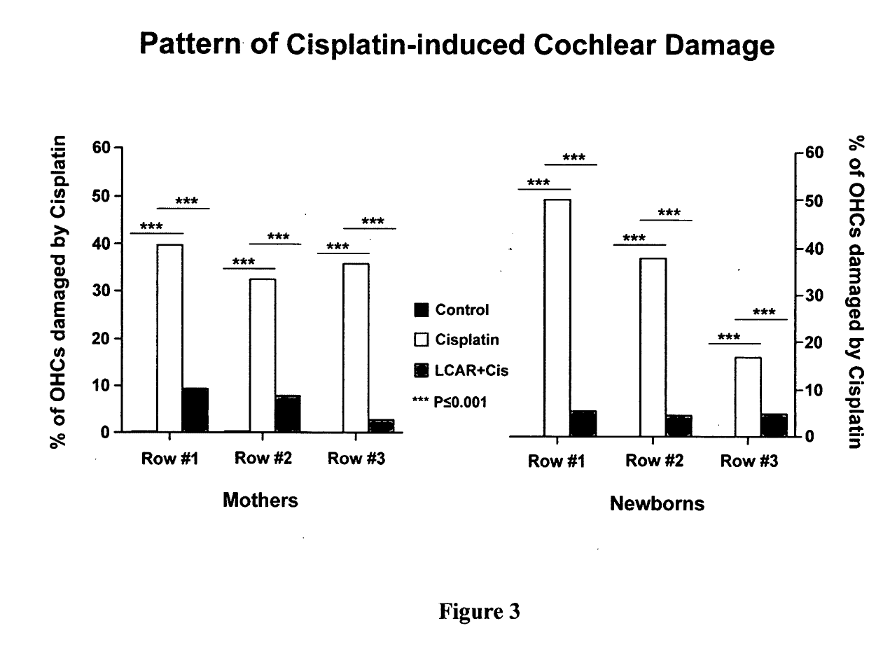

The ABR results described in FIG. 1 were independently corroborated by confocal microscopy of the organ of Corti in mothers and newborns. The confocal microscopy images in FIG. 2 show that the extensive cisplatin-induced damage to outer hair cells (OHCs) (FIG. 2B) was notably reduced in LCAR-supplemented guinea pigs (FIG. 2C). Evaluation of the cisplatin-induced cochlear damage indicate a significant loss of OHCs in the first and second turn of the cochlea of cisplatin-treated mothers and newborns which were not supplemented with LCAR (P≦0.01), but not in the apical turns (P≦0.08). LCAR supplementation attenuates this cisplatin-induced OHC damage, with only non-significant differences with the Control group (FIG. 2A). Interestingly, no significant cisplatin-induced inner hair cell (IHC) damage was observed in these experiments. The results of FIG. 2 are quantified in Table 1 and FIG. 3. Altogether these results indicate that LCAR reduces the damage caused to the cochlea caused by ototoxic agents.

TABLE 1
Cisplatin-induced cochlear damage

| | | | | Total % |
|---|---|---|---|---|
| | OHC (%) | | | (damaged/total |
| Group | Row #1 | Row #2 | Row #3 | OHCs) |
| Mothers | | | | |
| Control (n = 4) | 0.4 | 0.4 | 0 | 0.2 (2/812) |
| Cisplatin (n = 3) | 39.6 | 32.6 | 35.9 | 36.0 (297/824) |
| LCAR + Cisplatin | 9.5 | 7.9 | 2.7 | 6.7 (51/764) |
| (n = 4) | | | | |
| Newborns | | | | |
| Control (n = 14) | 0 | 0 | 0 | 0.0 (0/1832) |
| Cisplatin (n = 9) | 50.2 | 37.9 | 16.9 | 34.9 (443/1268) |
| LCAR + Cisplatin | 5.3 | 4.6 | 4.9 | 4.9 (139/2813) |
| (n = 17) | | | | |

### LCAR Supplementation Decreases Gentamicin-Induced Neonatal Mortality

LCAR deficiency frequently occurs in pregnant women and in premature neonates receiving parenteral alimentation. LCAR is required for fatty acid utilization, normal mitochondrial function, ATP formation, and intracellular detoxification. Conversely, LCAR depletion results in reduced energy metabolism, multiorgan dysfunction and even death, secondary to metabolic stress. There is, however, no available information regarding the possible impact of LCAR depletion and supplementation on mortality in metabolically stressed neonates.

Guinea pigs were used to evaluate the impact of prenatal LCAR supplementation on stress- and gentamicin-induced mortality in newborns because this animal model closely resembles perinatal human physiology. Pregnant guinea pigs (n=25) were divided in five groups. Those from group #1 did not receive any treatment or injection. In group #2, stress was induced by intraperitoneal injections of normal saline (once daily from days 51 to 57 of gestation). Guinea pigs in group #3 were injected with gentamicin (100 mg/kg once daily from days 51 to 57 of gestation). Finally, groups #4 and #5 were injected with gentamicin as described for group #3, but received LCAR supplementation in their water supply (1 mg/ml˜100 mg/kg/day) starting either 2 weeks prior (Group #4) or simultaneously with gentamicin (Group #5). Newborn mortality was defined as either stillborn or death within the first 48 hrs of life.

A total of 102 babies were born to 24 pregnant guinea pigs (a pregnant group #1 animal died and was excluded). No significant differences in neonatal mortality associated with stress (group #1, 11% vs. group #2, 31%) were found. Gentamicin alone increased neonatal mortality but not significantly at the 0.05 level (group #3, 54% vs. group #2, 31%; P≦0.08), although the effects of stress plus gentamicin were significant (group #3, 54% vs. group #1, 11%; P≦0.01). In contrast, neonatal mortality among animals exposed to gentamicin decreased in the two groups receiving daily LCAR supplementation, regardless of time of initiation (group #4, 15% vs. group #3, 54%; P≦0.01/group #5, 9% vs. group #3, 54%; P≦0.001). Altogether, these results indicate that LCAR supplementation reduces gentamicin-induced neonatal mortality in guinea pigs.

### LCAR Supplementation Prevents Gentamicin-Induced Sensorineural Hearing Loss and Cochlear Damage in Pregnant Guinea Pigs and Their Offspring

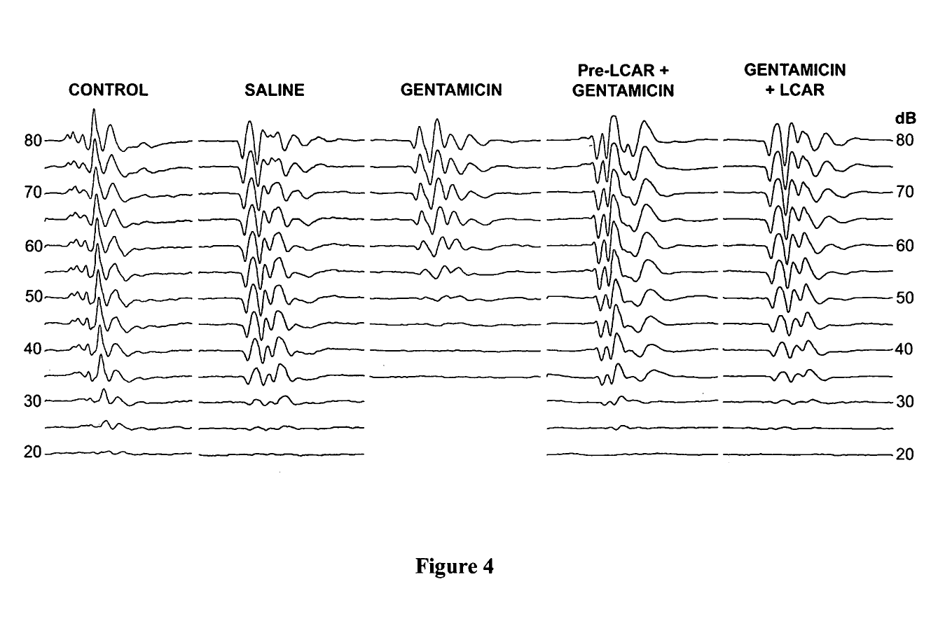

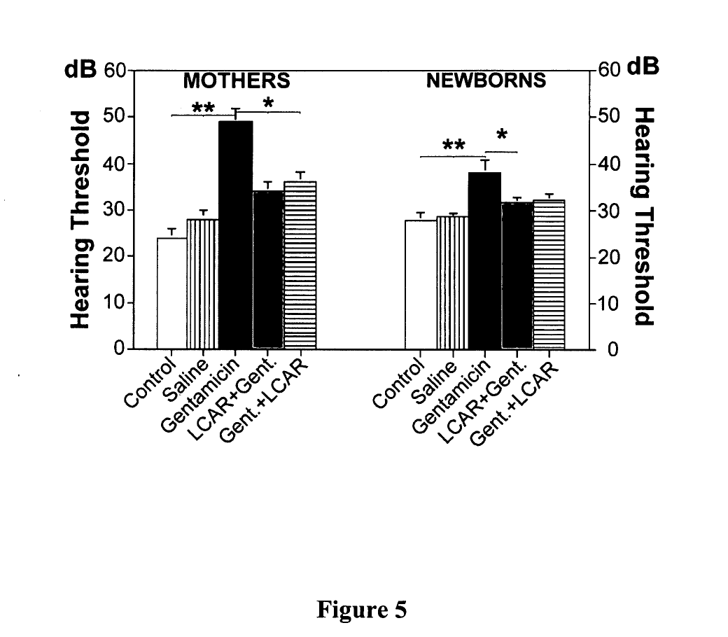

We investigated the hearing condition and cochlear integrity in the guinea pigs from the five experimental groups described in the above paragraph. Typical ABR recordings from newborn guinea pigs are shown in FIG. 4, while FIG. 5 describes the statistical results. No significant differences in hearing threshold were found between the two control groups (group #1—not injected vs. group #2—injected with normal saline). Gentamicin (group #3), in contrast, induced a significant increase in hearing threshold in respect to saline (group #2) in mothers (49±3 dB vs. 28±2; P≦0.01) and newborns (38±2 vs. 29±1; P≦0.01). Note, that in contrast to the effects associated with cisplatin, gentamicin appears to affect the mothers more than the pups (compare FIGS. 1C and 5). This may reflect the differences between experimental protocols, since gentamicin was injected during pregnancy whereas cisplatin (because of its toxicity) was injected to mothers and pups after delivery. Finally, LCAR supplementation—either from 28 days of pregnancy (group #4) or coincidental with gentamicin injections (group #5)—attenuates the ototoxic effects of gentamicin in both mothers and pups (FIG. 5).

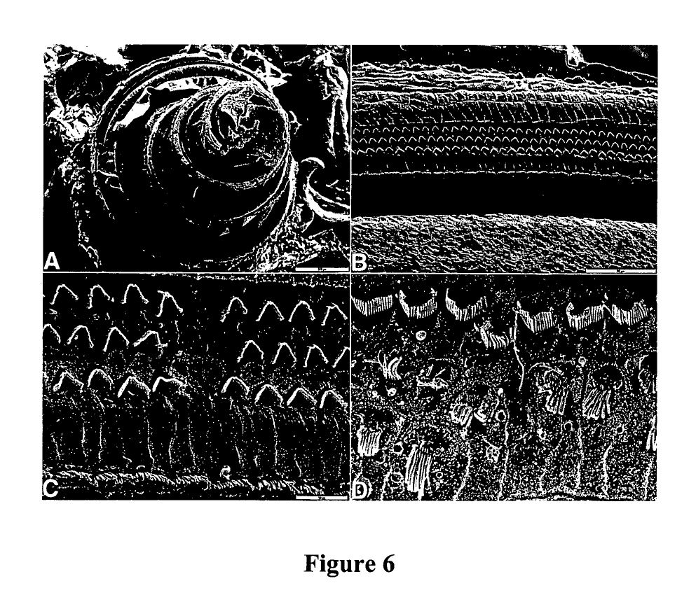

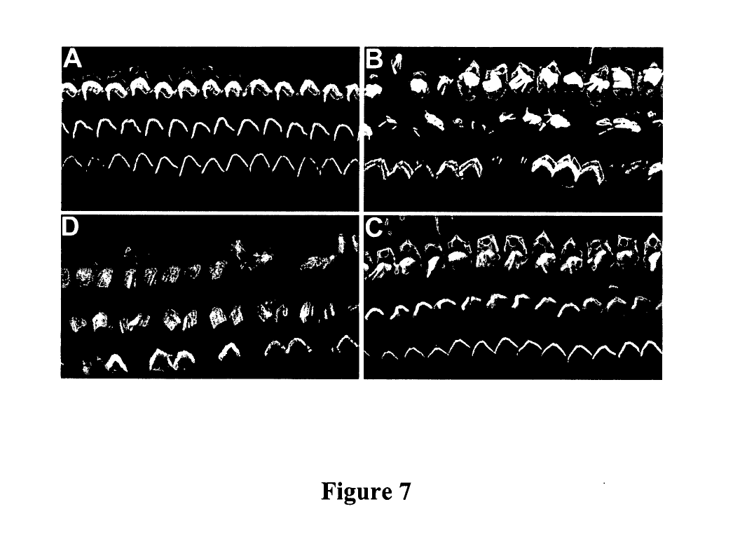

Confocal and electron microscopy studies indicate that gentamicin induces both sensory cell death and stereocilia disruption in mothers and newborn guinea pigs. As shown in FIG. 6A, full cochleas were investigated by scanning electron microscopy (SEM). Whereas in Control animals the three rows of OHCs look generally intact (FIG. 6B), cochleas from guinea pigs treated with gentamicin show “scars” where supporting cells replaced dead OHCs (FIG. 6C). In addition, stereocilia bundles look completely disorganized, with significant structural changes and even “giant” stereocilia (FIG. 6D). Our results indicate that LCAR supplementation, provided either prior or simultaneously with gentamicin, inhibit gentamicin-induced stereocilia disruption. In contrast, OHC death was significantly prevented only in animals supplemented with LCAR two weeks prior gentamicin treatment. FIG. 7 shows typical confocal images of intact OHCs' stereocilia bundles in control animals (FIG. 7A), disrupted in gentamicin-treated guinea pigs (FIG. 7B), and the effect of prior (FIG. 7C) or simultaneous (FIG. 7D) supplementation with LCAR. Table 2 describes the statistical results of gentamicin-induced OHC death.

TABLE 2
The percentage increase in outer hair cell death induced by
gentamicin, and a preventive effect by LCAR supplementation
against this effect

| | OHCs % (a/b) | | | |
|---|---|---|---|---|
| Groups | Row #1 | Row #2 | Row #3 | Total % (a/b) |
| Control | 0.1 | 0.2 | 0.2 | 0.2 |
| | (2/1820) | (3/1818) | (3/1818) | (8/3664) |
| Gentamicin | 0.6* | 0.9** | 0.8** | 0.7** |
| | (9/1516) | (13/1514) | (12/1523) | (34/4553) |
| LCAR + | 0.2 | 0.2 | 0.6* | 0.3 |
| Gentamicin | (4/2565) | (5/2565) | (15/2565) | (24/7695) |
| Gentamicin + | 0.8** | 0.4 | 1.0** | 0.7** |
| LCAR | (14/1709) | (7/1709) | (17/1709) | (38/5127) |

(a/b): (# damaged OHCs/# total OHCs);
*P ≦ 0.05;
**P ≦ 0.01

### LCAR Supplementation Decreases Noise-Induced Neonatal Mortality

Noise is considered an alpha-adrenergic stimulus that induces peripheral vasoconstriction, and has been described in clinical experiments as inducing short-term physiological reactions in the vegetative, endocrinological, neurological, and respiratory systems. If changes in circulation or endocrinological status take place, it can be expected that noise could have adverse effects on human and animal pregnancy. Several studies suggest that exposure to excessive noise during pregnancy may be associated with prematurity and intrauterine growth retardation. Women exposed to 80 dB for an 8-hour shift were at increased risk of preterm delivery. In a study involving 22,761 live births, women with self-reported noise exposure in health care jobs showed an increased risk of preterm delivery. In a case-control study of premature births among US nurses, constant noise was significantly associated with gestations of less than 37 weeks. Decreased birth weight has also been associated with noise exposure.

In order to evaluate noise-induced neonatal mortality and the potential impact of prenatal LCAR supplementation, six pregnant guinea pigs were divided into three groups of two animals each. Pregnant guinea pigs in groups #1 and #2 were exposed 4 hours a day during 4 consecutive days to broadband noise (100 Hz-10 kHz) at 95 SPL during days 50 to 53 of gestation. In addition, guinea pigs in group #2 received daily LCAR supplementation in their water supply (1 mg/ml˜100 mg/kg/day) from day 28 of gestation. Group #3, the control group, did not receive any treatment. Noise exposure was performed in a sound booth, with the animals in individual plastic cages placed on a wire shelf at ˜0.8 m above the floor. Since at this time of pregnancy guinea pigs spend most of the time resting with the abdomen in contact with the cage floor, the speakers were placed below the shelf, with the sound waves directed toward the cage's floor. Sound pressure levels were measured with a calibrated digital sound level meter (Radio Shack #33-2055) inside the plastic cages. Auditory brainstem response (ABR) evaluation before noise exposure showed no significant differences in hearing threshold between the six mothers (21±2 dB).

The experimental protocol assume normal delivery after 59-62 days of pregnancy. In fact, control animals delivered at days 59 and 61, respectively. Noise-exposed guinea pigs, however, delivered at day 54 (group #1) and days 54 and 55 (group #2). These results implicate noise exposure in the preterm deliveries. However, the small number of animals involved in this experiment did not allow us to prove this conclusively, as a ±5 day error in the estimation of gestational age is possible. Neonatal mortality—expressed as the number of stillborn or newborn death within the first 48 hrs of life over the total number of animals delivered—was 77% in group #1 (7/9), 9% in group #2 (1/11), and 0% in group #3 (0/9). These results strongly indicate that these levels of noise exposure increase neonatal mortality in guinea pigs, and that LCAR supplementation prevents this noxious effect.

### LCAR Supplementation Prevents Noise-Induced Sensorineural Hearing Loss in Pregnant Guinea Pigs and Their Offspring

Noise-induced cochlear damage and changes in hearing threshold were evaluated in mothers and newborns (ten days after delivery) in the three groups described in the above paragraph. We found moderate threshold shifts (˜9 dB) in mothers exposed to noise (group #1). In contrast, no changes were observed in LCAR supplemented animals. In newborns, we also observed a higher hearing threshold in noise-exposed animals than in those of the control group (36±2 dB group #1 and 30±1 dB group #2 vs. 22±1 dB group #3 control). These results suggest that noise exposure of pregnant guinea pigs has a noxious effect on the hearing of the offspring.

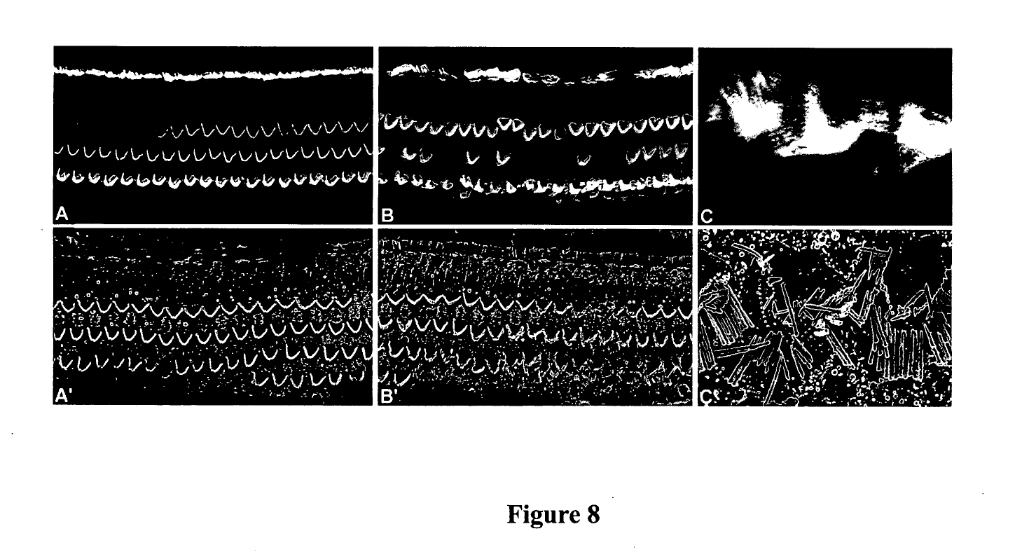

These results were corroborated by microscopic studies, where we observed extensive stereociliar disorganization and loss of hair cells in the organ of Corti of noise-exposed animals. See FIG. 8. Similar damage, but concentrated in smaller regions, was present in cochleas from LCAR-supplemented animals. The results indicate a significant decrease in cochlear damage associated with LCAR supplementation.

### LCAR Interferes with the Cisplatin- and Gentamicin-Activated Pathways that Induce Apoptosis

The HEI-Ve1 cell line was cloned from the vestibular organ of an Immortomouse™. HEI-Ve1 cells display many features characteristic of epithelial cells. They are polygonal and grow in a flat monolayer. They express the tight-junction proteins ZO-1 and occludin, cytokeratins 7 and 18, calmodulin, calbindin, calretinin and most importantly, Math 1 and myosin VIIa, markers of sensory hair cells. Expression of Math-1 and myosin VIIa indicates that HEI-Ve1 are sensory hair cell precursors.

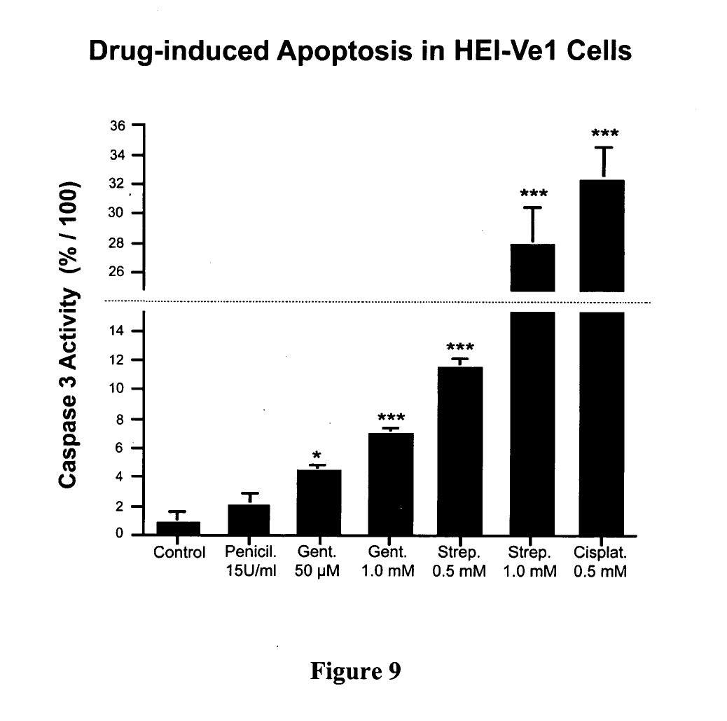

FIG. 9 shows the response of HEI-Ve1 cells to 24 hours of exposure to nontoxic antibiotic penicillin, ototoxic aminoglycoside antibiotics gentamicin and streptomycin, and pantoxic anti-tumor chemotherapeutic agent cisplatin. Light absorbance at λ=405 nm is used to measure caspase-3 activity, an early regulatory event in cell apoptosis.

The HEI-OC1 cell line was cloned from cochlear half-turn explants from of an Immortomouse™. Morphologically, HEI-OC1 cells have characteristics of epithelial cells, but do not resemble typical cochlear sensory cells. They are polygonal squamous cells with a dense perinuclear region containing a dense accumulation of multivesicular structures. These multivesicular structures were also observed in the rest of the cytoplasm, although at a lower density. HEI-OC1 cells co-express markers specific for both sensory and supporting cells. Expression of Math-1 and myosin VIIa by these cells indicate that HEI-OC1 cells are indeed sensory hair cell precursors.

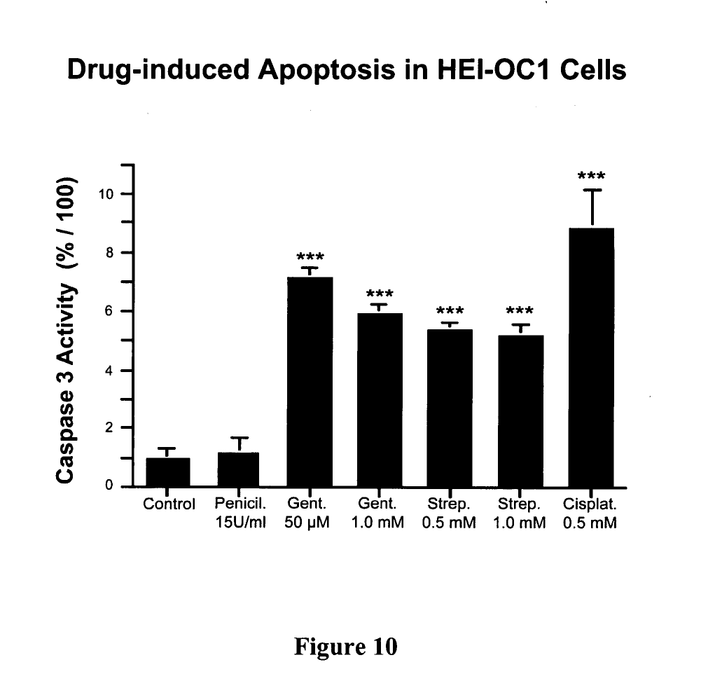

The response of HEI-OC1 cells to 24 hours of exposure to nontoxic antibiotic penicillin, ototoxic aminoglycoside antibiotics gentamicin and streptomycin, and pantoxic anti-tumor chemotherapeutic agent cisplatin is shown in FIG. 10. Light absorbance at λ=405 nm is used to measure caspase-3 activity, an early regulatory event in cell apoptosis.

Experiments performed with cisplatin show that cisplatin induces apoptosis in HEI-OC1 cells through mitochondrial pathways. Confluent HEI-OC1 cells were incubated with different doses of cisplatin (ranging from 25 μM to 500 μM) for 24 hours and then subjected to DNA Laddering and TUNEL assays. It was found that cisplatin induced apoptosis of HEI-OC1 cells in a dose- and time-dependent manner. Apoptosis, seen as a laddering pattern, was first detected at a cisplatin concentration of 100 μM and clearly visible for concentrations of 250 μM and 500 μM. HEI-OC1 cells were then incubated with 100 μM cisplatin for periods ranging from 6 hours to 24 hours. DNA laddering was only visible after 18-hour incubation with cisplatin. Apoptosis was confirmed using TUNEL (ApoAlert DNA Fragmentation Assay—Clontech, Palo Alto, Calif.).

Caspase-8 and caspase-9 assays (Clontech ApoAlert fluorescent kits) were used to clarify the apoptotic pathway triggered by cisplatin in HEI-OC1 cells. While little change in activity of caspase-8 was observed in cisplatin-treated cells, caspase-9 activity increased about five-fold after 12-hour incubation with 100 μM cisplatin. The addition of caspase-9 specific inhibitor LEHD-CCHO to the assay mixture completely prevented cell apoptosis, supporting the idea that cisplatin activates a caspase-9-mediated signaling pathway. Finally, immunofluorescence experiments demonstrating translocation of Cytochrome C from the mitochondria to the cytosol and Bax activation in cisplatin-treated cells, as well as changes in mitochondria permeability (detected by a Clonetech ApoAlert Mitochondrial Sensor Probe) further confirmed that cisplatin induces apoptosis through the activation of a mitochondrial signaling pathway in HEI-OC1 cells.

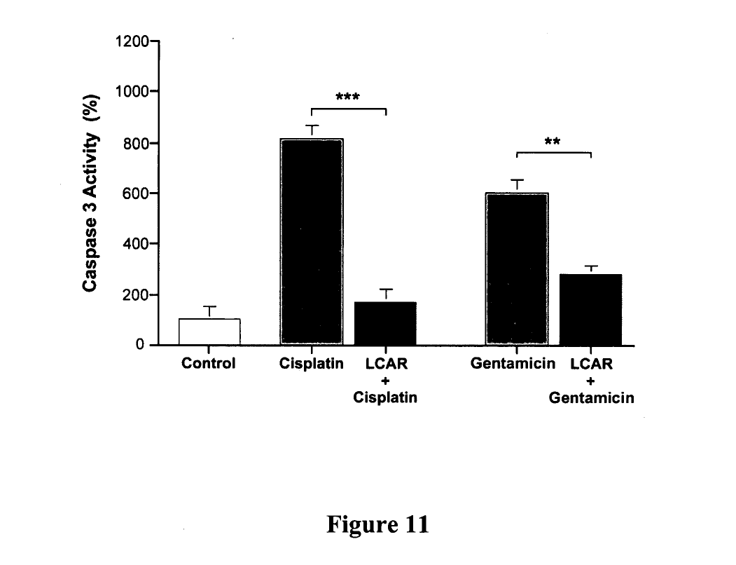

We further investigated whether LCAR was able to prevent cisplatin- and gentamicin-induced apoptosis in HEI-OC1 cells. Confluent cells were pre-incubated 48 hours with 2 mg/ml of LCAR, and then incubated 24 hours in a fresh medium containing 200 μM cisplatin (LCAR+cisplatin group) or 50 μM gentamicin. In addition, other samples of confluent HEI-OC1 cells were incubated either with cisplatin or gentamicin without pre-incubation with LCAR, or only with the culture medium (cisplatin, gentamicin, and control groups, respectively). We found that cisplatin- and gentamicin-induced cell apoptosis, as measured with the colorimetric CaspACE™ Assay (Promega, Madison, Wis.), was significantly inhibited by LCAR. See FIG. 11. These results indicate that HEI-OC1 cells are able to incorporate LCAR from the culture medium to replenish their intracellular storages, and that LCAR is able to interfere with the cisplatin- and gentamicin-activated pathways that induces HEI-OC1 apoptosis.

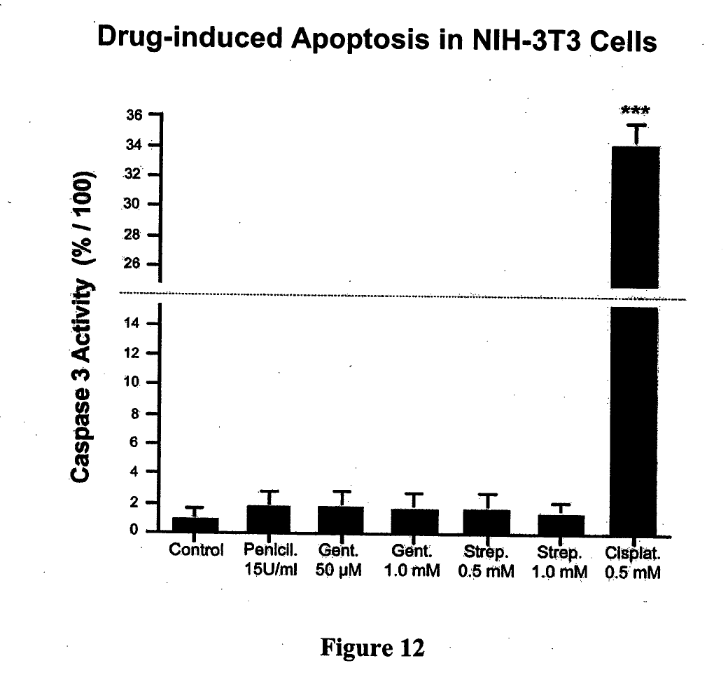

FIG. 12 shows the response of NIH3T3 cells to 24 hours of exposure to nontoxic antibiotic penicillin, ototoxic aminoglycoside antibiotics gentamicin and streptomycin, and pantoxic anti-tumor chemotherapeutic agent cisplatin. Light absorbance at λ=405 nm is used to measure caspase-3 activity, an early regulatory event in cell apoptosis. Apoptosis was induced in NIH3T3 fibroblasts as a control against which the results in FIGS. 9 and 10 are viewed.

We have recently demonstrated that cisplatin induces apoptosis in auditory cells by affecting the mitochondria. Cisplatin's effects include, but are not limited to an increase in the permeability of mitochondrial membranes and the release of Cytochrome C into the cytosol (Devarajan P. et al. 2002 Hear Res. 174:45-54). Since a similar pathway is activated by aminoglycoside antibiotics and other ototoxic drugs, the LCAR-induced restoration of the integrity of mitochondrial membranes and its ability to combat oxidative stress is envisioned to be increased by combining LCAR with other cofactors and proteins that enhance mitochondrial function.

We envision that a compound based on LCAR in combination with other molecules that promote mitochondrial function, will protect against drug- and noise-induced ototoxicity which are associated with the production of reactive oxygen species (ROS) and mitochondrial dysfunction which in turn can lead to apoptotic cell death. Additional constituents may include, but are not limited to, promoters of mitochondrial function, facilitators of oxidative phosphorylation and substances that combat oxidative stress. Since our results indicate that LCAR or its derivatives are effective either when provided prior to or during the exposure to harmful agents, the additional constituents recited above are contemplated to be delivered also prior or during the exposure to an ototoxic agent in order to reduce the damage caused by the latter. However, delivery of LCAR, or its derivatives, and the additional constituents recited above after the exposure to an ototoxic agent is also contemplated.

While the present invention has been described in some detail for purposes of clarity and understanding, one skilled in the art will appreciate that various changes in form and detail can be made without departing from the true scope of the invention. All figures, tables, and publications, referred to above, are hereby incorporated by reference.

1. A method of preventing or treating a hearing loss induced in a pregnant mammal and/or its offspring by exposure to an ototoxic agent during the perinatal period, comprising administering to said pregnant mammal and/or its offspring during the perinatal period an amount of a pharmaceutical composition comprising L-carnitine or a derivative thereof, said amount being sufficient to prevent or treat the hearing loss induced by exposure to said ototoxic agent.

2. The method of claim 1, wherein said ototoxic agent is an antibiotic.

3. The method of claim 2, wherein said antibiotic is an aminoglycoside.

4. The method of claim 1, wherein said ototoxic agent is a chemotherapeutic agent.

5. The method of claim 1, wherein said ototoxic agent is sound.

6. The method of claim 1, wherein said perinatal mammal is exposed to said ototoxic agent in utero.

7. The method of claim 1, wherein said administering is prenatal.

8. The method of claim 1, wherein said administering is postnatal.

9. The method of claim 1, wherein said administering to said perinatal mammal further comprises administering to a pregnant mammal and its offspring.

10. The method of claim 1, wherein said mammal is a fetus.

11. The method of claim 1, wherein said mammal is a newborn.

12. The method of claim 1, wherein said administering is performed before exposure to said ototoxic agent.

13. The method of claim 1, wherein said administering is performed concurrent with exposure to said ototoxic agent.

14. The method of claim 1, wherein said administering is performed after exposure to said ototoxic agent.

15. The method of claim 1, wherein said derivative of L-carnitine is an ester.

16. The method of claim 15, wherein said ester is acetyl-L-carnitine.

17. A method of preventing noise-induced neonatal mortality in a mammal, comprising administering to said mammal an amount of a pharmaceutical composition comprising L-carnitine or derivatives thereof, wherein said amount is sufficient to prevent the noise-induced neonatal mortality.

18. A method of preventing antibiotic-induced neonatal mortality in a mammal, comprising administering to said mammal an amount of a pharmaceutical composition comprising L-carnitine or derivatives thereof, wherein said amount is sufficient to prevent the antibiotic-induced neonatal mortality.

19. A method of preventing or treating ototoxin-induced cell oxidative stress and/or apoptosis in a mammal, comprising administering to said mammal an amount of a pharmaceutical composition comprising L-carnitine or derivatives thereof, wherein said amount is sufficient to prevent or treat the ototoxin-induced cell oxidative stress and/or apoptosis.

20. The method of claim 19, wherein said cell is located in the vestibular organ.

21. The method of claim 19, wherein said cell is located in the organ of Corti.

22. The method of claim 19, wherein said derivative is an ester of L-carnitine.

23. The method of claim 22, wherein said ester is acetyl-L-carnitine.

24. The method of claim 19, wherein said ototoxin is an antibiotic.

25. The method of claim 19, wherein said ototoxin is an aminoglycoside.

26. The method of claim 19, wherein said ototoxin is a chemotherapeutic compound.

27. The method of claim 19, wherein said ototoxin is sound.
